SEQUENCE LISTING (10 sequences)

SEQ ID NO: 1 - protein, 228 aa, Arabidopsis thaliana
MGRGKIVIQRIDDSTSRQVTFSKRRKGLIKKAKELAILCDAEVGLIIFSSTGKLYDFASSSMKSVIDRYNKSKIEQQQLLNPASEVKFWQREAAVLRQELHALQENHRQMMGEQLNGLSVNELNSLENQIEISLRGIRMRKEQLLTQEIQELSQKRNLIHQENLDLSRKVQRIHQENVELYKKAYMANTNGFTHREVAVADDESHTQIRLQLSQPEHSDYDTPPRANE

SEQ ID NO: 2 - DNA, 687 bp, Arabidopsis thaliana
atgggaagagggaagattgtgatccaaaggatcgatgattcaacgagtagacaagtcactttctccaaacgaagaaagggccttatcaagaaagccaaagagctagctattctctgtgatgccgaggtcggtctcatcatcttctctagcaccggaaagctctatgactttgcaagctccagcatgaagtcggttattgatagatacaacaagagcaagatcgagcaacaacaactattgaaccccgcatcagaagtcaagttttggcagagagaagctgctgttctaagacaagaactgcatgctttgcaagaaaatcatcggcaaatgatgggagaacagctaaatggtttaagtgttaacgagctaaacagtcttgagaatcaaattgagataagtttgcgtggaattcgtatgagaaaggaacaactgttgactcaagaaatccaagaactaagccaaaagaggaatcttattcatcaggaaaacctcgatttatctaggaaagtacaacggattcatcaagaaaatgtggagctctacaagaaggcttatatggcaaacacaaacgggtttacacaccgtgaagtagctgttgcggatgatgaatcacacactcagattcggctgcaactaagccagcctgaacattccgattatgacactccaccaagagcaaacgaataa

SEQ ID NO: 3 - DNA, 32 bp, Artificial
  primer
cacccacagcaaagataaacacacacaattac

SEQ ID NO: 4 - DNA, 30 bp, Artificial
  primer
caattttatcctctaattgaatctcctctg

SEQ ID NO: 5 - DNA, 28 bp, Artificial
  primer
caccatgggaagagggaagattgtgatc

SEQ ID NO: 6 - DNA, 25 bp, Artificial
  primer
ttattcgtttgctcttggtggagtg

SEQ ID NO: 7 - DNA, 22 bp, Artificial
  primer
atgtggagctctacaagaaggc

SEQ ID NO: 8 - DNA, 22 bp, Artificial
  primer
ttcgtttgctcttggtggagtg

SEQ ID NO: 9 - DNA, 23 bp, Artificial
  primer
ccaagatccaggacaaagaagga

SEQ ID NO: 10 - DNA, 22 bp, Artificial
  primer
tggagacgagcataacacttgc

(19) United States
(12) Patent Application Publication
(10) Pub. No.: US 2011/0093985 A1
(43) Pub. Date: Apr. 21, 2011

(54) TRANSCRIPTION FACTOR FOR PROMOTING LATERAL ROOT GROWTH UNDER NITROGEN-LIMITING CONDITIONS

(75) Inventors: AKINORI SUZUKI, KANAGAWA (JP); HIDEKI TAKAHASHI, KANAGAWA (JP)

(73) Assignee: RIKEN, WAKO-SHI (JP)

(21) Appl. No.: 12/893,954

(22) Filed: Sep. 29, 2010

Related U.S. Application Data
(60) Provisional application No. 61/246,962, filed on Sep. 29, 2009.

(51) Int. Cl. C12N 15/82; A61K 36/31; C07H 21/04; A01H 5/00; A01G 1/00

(52) U.S. Cl. 800/290; 530/370; 536/23.6; 800/298; 435/320.1; 47/58.1 R

(57) ABSTRACT

This invention provides a transcription factor AGL21 (AGAMOUS-LIKE 21), which can positively regulate the lateral root growth of a plant when the external supply of N is limited, a homologue thereof or a mutant thereof, gene encoding thereof and a transformed plant with the gene.

### CROSS-REFERENCE

This application claims the benefit of U.S. Provisional Application No. 61/246,962 filed Sep. 29, 2009, the contents of which are incorporated by reference herein in their entirety.

### FIELD OF THE INVENTION

This invention relates to a transcription factor which can promote the lateral root growth in a plant under nitrogen-limiting conditions and a method for growing a plant on land with limiting nitrogen source using transgenic plants containing a polynucleotide encoding the transcription factor or the progeny thereof.

### BACKGROUND OF THE INVENTION

Nitrogen (N) is the mineral nutrient required in greatest amounts for plant growth. Among the different N sources in the environment, nitrate and ammonium are the major inorganic forms of N available from the soil (Marschner, 1995). Since nitrate is easily leached from the soil by rainfall, nitrate concentrations can be highly variable. Ammonium is a less mobile form of N because of the strong cation exchange capacity of the soil. Responding to the changes in nitrate availability, plants express specific transport systems to efficiently absorb nitrate from the rhizosphere (Crawford and Glass, 1998; Daniel-Vedele et al., 1998; Forde, 2000; Williams and Miller, 2001). Plants are also able to modify their root architecture to take account of heterogeneously distributed supplies of N, particularly nitrate, from the environment (Drew, 1975; Robinson, 1994, Forde and Lorenzo, 2001). The mechanisms for modifying root architecture involve the intrinsic pathways that determine organ and cell identities, and the response pathways that modulate developmental processes depending on specific environmental signals (Malamy, 2005). Recent studies in Arabidopsis (Arabidopsis thaliana) have identified several signaling components involved in nitrate regulation of root development.

Many plants typically respond to the presence of nitrate-rich patches of soil by proliferating their roots within the patch (Drew, 1975; Robinson, 1994). The ANR1 MADS-box transcription factor was identified as a key element controlling the elongation of lateral roots in response to localized supplies of nitrate (Zhang and Forde, 1998). In a split-root culture, NRT1.1 nitrate transporter was also shown to be required for proliferation of secondary laterals on high-nitrate patches, and appeared to perform a role in nitrate signaling pathway regulating the expression of ANR1 (Remans et al., 2006a). The proposed sensory functions of ANR1 and NRT1.1 are thought to be relevant to the ability of the plant to capture heterogeneously distributed supplies of nitrate from the soil. NRT1.1 was also shown to be required for nitrate to alleviate the inhibitory effect of glutamate on primary root growth (Walch-Liu and Forde, 2008). More recently, a calcineurin B-like interacting protein kinase (CIPK8) was reported to be involved in nitrate regulation of nitrate-inducible genes, including NRT1.1, and in the regulation of primary root growth (Hu et al., 2009).

In addition to these responses to the external nitrate supply, lateral root growth and development is also responsive to endogenous signals related to the N status of the plant. A screen for mutants in the regulation of lateral root initiation identified a high-affinity nitrate transporter, NRT2.1, as involved in restricting the formation of lateral roots in high sucrose/low N conditions (Malamy and Ryan, 2001; Little et al., 2005). Nitrate in excess relative to carbon inhibited the early development of lateral roots (Zhang et al., 1999), while nitrate limitation led to an increase in mean lateral root length, particularly when the NRT2.1 and NRT2.2 genes were inactive (Remans et al., 2006b).

### SUMMARY OF THE INVENTION

The objects of the present invention are to isolate a gene which promotes the lateral root growth in a plant under nitrogen-limiting conditions and to provide a plant which can grow on land with limiting nitrogen source.

The present inventors have conducted concentrated studies in order to attain the above objects. As a result, they have isolated AGL21 (AGAMOUS-LIKE 21), a member of the ANR1-family MADS-box transcription factors, and characterized its relevance to the root morphological response in low nitrate environment. The present invention has been completed based on such findings.

Specifically, the present invention includes the following aspect.

In one aspect, the present invention relates to a MADS-box transcription factor which can promote the lateral root growth in a plant under nitrogen-limiting conditions. The plant preferably belongs to Brassicaceae, Fabaceae, Poaceae, Solanaceae, Vitaceae, Euphorbiaceae, Salicaceae or Myrtaceae.

In one embodiment of this aspect, the MADS-box transcription factor comprises any one of the following amino acid sequences (a) to (c):

(a) an amino acid sequence defined in SEQ ID NO: 1

(b) an amino acid sequence comprising one or more amino acid deletions, substitutions and/or additions in the amino acid sequence defined in SEQ ID NO: 1

(c) an amino acid sequence sharing more than 50% homology with the amino acid sequence defined in SEQ ID NO: 1

In another aspect, the present invention relates to a polynucleotide encoding the MADS-box transcription factor of the present invention.

In further aspect, the present invention relates to a vector comprising said polynucleotide. The vector includes Ti plasmid or binary plasmid.

In further aspect, the present invention relates to a transgenic plant or a progeny thereof, comprising said polynucleotide or said expression vector.

In one embodiment, the expression of the polynucleotide of the present invention is enhanced in the transgenic plant or the progeny thereof.

In another embodiment, the region wherein the expression of said polynucleotide is enhanced is root.

In still further aspect, the present invention relates to a method for growing a plant on land with limiting nitrogen source using said transgenic plant or the progeny thereof.

In further aspect, the present invention relates to a method for producing a plant in which the lateral root growth is promoted, comprising the following steps:

introducing said polynucleotide or said vector into a plant, a cell or tissue thereof;

optionally, culturing the cell or tissue to reproduce plant bodies; and

selecting a plant with promoted growth of lateral roots from among the plant bodies, which are cultivated under nitrogen-limiting conditions.

### BRIEF DESCRIPTION OF THE DRAWINGS

FIG. 1 shows knockout of AGL21 in Arabidopsis.

FIG. 1A shows locations of Ds and dSpm in AGL21. Boxes and lines indicate exons and introns, respectively. Bar=1 kb. Ds and dSpm elements are not drawn in exact sizes.

FIG. 1B shows quantification of AGL21 transcript levels in agl21-1 and agl21-2 mutants, and their corresponding wild-type ecotypes, Nossen (No) and Columbia (Col). Biological triplicate samples of RNA were extracted from the roots of 8-day-old seedlings grown on 0.1 mM nitrate medium. The mRNA levels are indicated as values relative to those of ecotype Nossen (means±SEs).

FIG. 2 shows the short lateral root phenotypes of agl21 mutants.

FIG. 2A shows scanned images of 8-day-old seedlings of agl21-1 and agl21-2 mutants, and their corresponding wild-type ecotypes, Nossen (No) and Columbia (Col). Plants were grown on 0.1 mM nitrate medium. Bars=1 cm.

FIG. 2B shows total lateral root length.

FIG. 2C shows total lateral root number.

FIG. 2D shows primary root length. Values indicate means±SEs (n=10).

FIG. 3 shows overexpression of AGL21 restores the agl21 phenotypes.

FIG. 3A shows scanned images of 8-day-old seedlings of wild-type Nossen (No), agl21-1 mutant, AGL21 overexpressors in agl21-1 background (AGL21/agl21-1 #20 and #10), and their null segregants (Null/agl21-1 #20 and #10). Plants were grown on 0.1 mM nitrate medium. Bars=1 cm.

FIG. 3B shows total lateral root length.

FIG. 3C shows total lateral root number.

FIG. 3D shows primary root length. Values indicate means±SEs (n=12-17).

FIG. 4 shows nitrate dependency of the phenotypes of agl21 mutant and AGL21 overexpressors.

FIG. 4A shows scanned images of 8-day-old seedlings of wild-type Nossen (No), agl21-1 mutant, and AGL21 overexpressors in agl21-1 background (AGL21/agl21-1 #20 and #10) grown on 0, 0.1 and 1 mM nitrate medium. Bars=1 cm.

FIG. 4B shows total lateral root length which was quantified for the 8-day-old seedlings grown on 0, 0.01, 0.03, 0.1, 0.3 or 1 mM nitrate medium.

FIG. 4C shows total lateral root number which was quantified for the 8-day-old seedlings grown on 0, 0.01, 0.03, 0.1, 0.3 or 1 mM nitrate medium.

FIG. 4D shows primary root length which was quantified for the 8-day-old seedlings grown on 0, 0.01, 0.03, 0.1, 0.3 or 1 mM nitrate medium. Values indicate means±SEs (n=12-20).

FIG. 5 shows lateral root growth in the presence of alternative nitrogen source.

FIG. 5A shows scanned images of 8-day-old seedlings of wild-type Nossen (No), agl21-1 mutant, and AGL21 overexpressors in agl21-1 background (AGL21/agl21-1 #20 and #10) grown on nitrate-less medium containing 0.1 mM Gln. Bars=1 cm.

FIG. 5E shows the phenotypes of the same plant lines as in FIG. 5A. The phenotypes were analyzed on the medium containing 0.1 mM Gln and 0.1 mM nitrate. Bars=1 cm.

FIG. 5B and FIG. 5F show total lateral root length which was quantified for the 8-day-old seedlings.

FIG. 5C and FIG. 5G show total lateral root number which was quantified for the 8-day-old seedlings.

FIG. 5D and FIG. 5H show primary root length which were quantified for the 8-day-old seedlings. Values indicate means±SEs (n=11-20).

FIG. 6 shows localization of AGL21 expression.

FIG. 6A shows longitudinal section of primary root tip. Bar=50 μm.

FIG. 6B shows longitudinal section of lateral root tip. Bar=50 μm.

FIG. 6C shows close-up view of B in the tip region. Bar=20 μm.

FIG. 6D shows cross section of lateral root. Bar=20 μm. Green and red indicate fluorescent signals of GFP and propidum iodide, respectively. The cross sections were constructed from Z-series confocal images. C, cortex; CRC, columella root-cap; En, endodermis; Ep, epidermis; LRC, lateral root-cap.

### DETAILED DESCRIPTION OF THE INVENTION

### 1. A Transcription Factor Promoting the Lateral Root Growth Under Nitrogen-Limiting Conditions

The protein according to the present invention is a MADS-box transcription factor, which can promote the lateral root growth in a plant under nitrogen-limiting conditions. Preferably, the protein is AGL21, which is a member of the ANR1 clade of MADS-box transcription factors. The ANR1 clade contains four proteins, ANR1, AGL21, AGL16 and AGL17 and is notable among other MADS-box clades in that its members are preferentially expressed in roots (Gan et al., 2005). AGL21, like ANR1, is a positive regulator of lateral root growth but its role is most significant under N-limiting conditions.

AGL21 comprises any one of the following amino acid sequences (a) to (c).

(a) An amino acid sequence of Arabidopsis thaliana AGL21

First, the amino acid sequence of AGL21 is defined in SEQ ID NO: 1, which is AGL21 of Arabidopsis thaliana.

(b) An amino acid sequence comprising mutation(s) in the amino acid sequence of A. thaliana AGL21

Second, the amino acid sequence of AGL21 comprises mutation(s) in the amino acid sequence defined in SEQ ID NO: 1. The term “mutation” comprises one or more, preferably one or several, deletions, substitutions or additions in the amino acid sequence of AGL21 defined in SEQ ID NO: 1. The number of the amino acid residues that may be deleted, substituted, or added refers to the number that can be deleted, substituted, or added by a conventional method of preparing a mutant protein, such as site-directed mutagenesis. Such number is preferably 1 or more. For example, 1 to 10, and preferably 1 to 5, amino acid residues may be deleted from the amino acid sequence as shown in any of SEQ ID NO: 1, and preferably 1 to 5, amino acid residues may be added to the amino acid sequence as shown in any of SEQ ID NO: 1; or 1 to 10, and preferably 1 to 5, amino acid residues may be substituted with other amino acid residues in the amino acid sequence as shown in any of SEQ ID NO: 1.

The mutation may include either naturally occurring mutations or artificial mutations. Where the mutation is of a protein or polypeptide, preferable substitutions are conservative substitutions, which are substitutions between amino acids similar in properties such as structural, electric, polar, or hydrophobic properties. For example, the substitution can be conducted between basic amino acids (e.g., Lys, Arg, and His), or between acidic amino acids (e.g., Asp and Glu), or between amino acids having non-charged polar side chains (e.g., Gly, Asn, Gln, Ser, Thr, Tyr, and Cys), or between amino acids having hydrophobic side chains (e.g., Ala, Val, Leu, Ile, Pro, Phe, and Met), or between amino acids having branched side chains (e.g., Thr, Val, Leu, and Ile), or between amino acids having aromatic side chains (e.g., Tyr, Trp, Phe, and His).

The amino acid residues(s) can be deleted, added, or substituted through modifying the gene encoding the protein by a technique known in the art. Mutation can be introduced into a gene via conventional techniques such as the Kunkel method or the Gapped duplex method. The mutation may also be introduced using a mutagenesis kit, such as a Mutant-K (Takara) or Mutant-G (Takara), utilizing site-directed mutagenesis or the Takara LA PCR in vitro Mutagenesis series kit (Takara).

(c) An amino acid sequence of homologue protein of A. thaliana AGL21

Third, the amino acid sequence of AGL21 may have a homology to the amino acid sequence defined in SEQ ID NO: 1.

As used herein, the term “homologue protein” means a protein from any plant other than the angiosperm Arabidopsis thaliana, in which the protein comprises an amino acid sequence homologous to that of AGL21 protein and stimulates the lateral root growth.

The homologue proteins of the AGL21, whose amino acid sequences have at least 20%, preferably at least 50%, more preferably at least 80%, yet more preferably at least 90-98% identity to the amino acid sequence of SEQ ID NO: 1, and having an activity of promoting the lateral root growth under nitrogen-limiting conditions

Phylogenetically, AGL21 is most closely related to AGL17 (Parenicova et al., 2003), but its spatial pattern of expression in the root was reported to be more similar to ANR1 than to AGL17, suggesting that ANR1 and AGL21 may have a degree of functional redundancy (Burgeff et al., 2002). The observation that ANR1 down-regulated lines have a distinctive root phenotype (Zhang and Forde, 1998) indicates that any functional redundancy between the two genes is not complete, but it is nevertheless possible that they have overlapping or related roles in the regulation of root development.

The amino acid sequence of homologue proteins of AGL21, that is, AGL21 orthologous protein, can be searched is available from known databases such as NCBI GenBank (USA), EMBL (Europe), etc. Some A. thaliana AGL21 orthologous proteins have been isolated in many plants such as Vitis venifera (for example, Accession Number: XP_{—}002283694, XP_{—}002273556 and XP_{—}002265503), Populus trichocarpa (for example, Accession Number: XP_{—}002307325, XP_{—}002302361, XP_{—}002313958, XP_{—}002300317 and XP_{—}002300316), Ricinus communis (for example, Accession Number: XP_{—}002527350 and XP_{—}002518331), Zea mays (for example, Accession Number: NP_{—}001104926) and Oryza sativa (for example, Accession Number: Os02g0579600, Os02g0731200, Os04g0304400 and Os08g0431900). These orthologous have more than 50% identity with the amino acid sequence of AGL21 defined in SEQ ID NO: 1.

As used herein, the term “nitrogen-limiting conditions” means the conditions that there is limited amount of the nitrogen source such as nitrate and ammonium in the soil or the culture medium. The scope of the “limited amount” is defined as a range of nitrogen concentration from 0 to 0.2 mM, preferably from 0 to 0.1 mM, more preferably from 0 to 0.05 mM, and most preferably from 0 to 0.03 mM.

The term “lateral root” means the branches of roots initiated from the primary root, and higher order branches of roots initiated from that lateral root.

As used herein, the wording “promote(s) the lateral root growth”, “promoted growth of lateral roots” or “the lateral root growth is promoted” means increasing the number and/or length of visible lateral roots when compared with the wild type.

### 2. A Polynucleotide Encoding the Transcription Factor Promoting the Lateral Root Growth Under Nitrogen-Limiting Conditions

The polynucleotide according to the present invention encodes aforementioned MADS-box transcription factor. Preferably, the polynucleotide is AGL21 gene. AGL21 gene comprises any one of the genes encoding aforementioned amino acid sequences (a) to (c) of AGL21 protein. Specifically, the polynucleotide of the present invention may include a gene encoding AGL21 of A. thaliana, whose nucleotide sequence is shown in SEQ ID NO: 2, or a gene encoding a protein consisting of an amino acid sequence having 80% or higher homology to the amino acid sequence as shown in SEQ ID NO: 1 and having an activity of promoting lateral root growth under nitrogen-limiting conditions. The aforementioned 80% or higher homology preferably refers to homology of 85% or higher, more preferably to homology of 90% or higher, and most preferably to homology of 95% or higher. Sequence identity can be determined via a FASTA or BLAST search. The polynucleotide of the present invention may also include a gene having nucleotide sequences capable of hybridizing with a nucleotide sequence complement to the nucleotide sequence of SEQ ID NO: 2 under stringent conditions, wherein the nucleotide sequences having an activity of promoting lateral root growth under nitrogen-limiting conditions. As used herein, the term “stringent conditions” refers to conditions under which what is called a specific-hybrid is formed but a non-specific hybrid is not formed. For example, under such conditions, complementary strands of DNA consisting of a highly homologous nucleic acid, i.e., DNA consisting of a nucleotide sequence exhibiting 80% or higher, preferably 85% or higher, more preferably 90% or higher, and most preferably 95% or higher homology to the nucleotide sequence, hybridize, but complementary strands of a nucleic acid having homology lower than the aforementioned level do not hybridize. More specific conditions are constituted by a sodium salt concentration of 15 mM to 750 mM, and preferably 50 mM to 750 mM, and more preferably 300 mM to 750 mM, and a temperature of 25° C. to 70° C., preferably 50° C. to 70° C., and more preferably 55° C. to 65° C., and a formamide concentration of 0% to 50%, preferably 20% to 50%, and more preferably 35% to 45%. Under stringent conditions, further, the filter is washed after hybridization generally at a sodium salt concentration of 15 to 600 mM, preferably 50 to 600 mM, and more preferably 300 to 600 mM and a temperature of 50° C. to 70° C., preferably 55° C. to 70° C., and more preferably 60° C. to 65° C.

A person skilled in the art can readily obtain such homolog genes with reference to, for example, Molecular Cloning (Sambrook, J. et al., Molecular Cloning: A Laboratory Manual 2nd ed., Cold Spring Harbor Laboratory Press, 10 Skyline Drive Plainview, N.Y., 1989). Also, homology of the above sequences can be determined via a FASTA or BLAST search.

The AGL21 gene used in the present invention can be obtained as a nucleic acid fragment via PCR amplification with the use of primers designed based on the nucleotide sequence information and nucleic acids as templates obtained from a cDNA library, genomic DNA library, or the like. Also, the AGL21 gene can be obtained as a nucleic acid fragment via hybridization using the nucleic acid obtained from the library as a template and a DNA fragment, which is part of the AGL21 gene, as a probe. Alternatively, the AGL21 gene may be synthesized as a nucleic acid fragment via various techniques of nucleic acid synthesis, such as chemical synthesis, known in the art.

### 3. Recombinant Vector

The recombinant vector according to the present invention that is used for plant transformation can be constructed by introducing the AGL21 gene (hereafter, this may be referred to as “the target gene”) into an adequate vector. For example, pBI, pPZP, and pSMA vectors that can introduce the target gene into a plant via Agrobacterium are preferably used. A pBI binary vector or intermediate vector is particularly preferable, and examples thereof include pBI121, pBI101, pBI101.2, and pBI101.3. A binary vector is a shuttle vector that can be replicated in E. coli and in Agrobacterium. When Agrobacterium containing a binary vector is allowed to infect plants, DNA in the portion sandwiched between border sequences consisting of the LB sequence and the RB sequence on the vector can be incorporated into the plant nuclear DNA. In contrast, a pUC vector can be used to directly introduce a gene into plants. Examples thereof include pUC18, pUC19, and pUC9 vectors. Plant virus vectors, such as cauliflower mosaic virus (CaMV), bean golden mosaic virus (BGMV), and tobacco mosaic virus (TMV) vectors, can also be used.

When a binary vector plasmid is used, the target gene is inserted between the border sequences (LB and RB sequences) of the binary vector, and this recombinant vector is then amplified in E. coli. Subsequently, the amplified recombinant vector is introduced into Agrobacterium tumefaciens GV3101, C58, LBA4404, EHA101, EHA105, or the like or Agrobacterium rhizogenes LBA1334 via electroporation or other means, and the aforementioned Agrobacterium is used for genetic transformation of plants.

The three-member conjugation method (Nucleic Acids Research, 12:8711, 1984) may also be used in addition to the method described above to prepare an Agrobacterium to infect plants containing the target gene. Specifically, plasmid-containing E. coli comprising the gene of interest, helper plasmid-containing E. coli (e.g., pRK2013), and an Agrobacterium are mixed and cultured on a medium containing rifampicin and kanamycin. Thus, a zygote Agrobacterium to infect plants can be obtained.

In order to insert the target gene into a vector, for example, a method may be employed in which the purified DNA is cleaved with an appropriate restriction enzyme and then inserted into the restriction site or the multi-cloning site of an appropriate vector DNA for ligation to the vector.

The target gene needs to be incorporated into a vector in a manner such that functions of the gene are exhibited. A promoter, an enhancer, a terminator, or a replication origin used for binary vector system (e.g., a replication origin derived from a Ti or Ri plasmid), a selection marker gene, or the like can be ligated to the vector at a site upstream, inside, or downstream of the target gene.

The “promoter” may or may not be derived from plants, as long as the DNA can function in plant cells and can induce expression in a specific plant tissue or during a specific growth phase. Specific examples thereof include a cauliflower mosaic virus (CaMV) 35S promoter, a nopalin synthase gene promoter (Pnos), a maize ubiquitin promoter, a rice actin promoter, and a tobacco PR protein promoter.

An example of an enhancer is an enhancer region that is used for improving the expression efficiency of the target gene and that comprises the upstream sequence in the CaMV 35S promoter.

Any terminator can be used as long as it can terminate transcription of the gene transcribed by a promoter. Examples thereof include a nopalin synthase (NOS) gene terminator, an octopine synthase (OCS) gene terminator, and a CaMV 35S RNA gene terminator.

Examples of a selection marker gene include an ampicillin resistant gene, a neomycin resistant gene, a hygromycin resistant gene, a bialaphos resistant gene, and a dihydrofolate reductase gene.

The selection marker gene and the target gene may be ligated to the same plasmid to prepare a recombinant vector as described above. Alternatively, a recombinant vector that is obtained by ligating the selection marker gene to a plasmid may be prepared separately from a recombinant vector that is obtained by ligating the target gene to a plasmid. When recombinant vectors are separately prepared, both vectors are cotransfected into a host.

### 4. Transgenic Plant and Method for Preparing the Same

The transformed plant of the invention is characteristic of having promotion of lateral root growth under nitrogen-limiting conditions. This characteristic of the plant is achieved by over-expressing a foreign (or exogenous) DNA coding for protein AGL21 or a homologue thereof in the plants.

As used herein, the term “over-expressing”, “over-expressed” or “over-expression” means that an expression level of the AGL21 protein or homologue proteins thereof in the transformed plant of the invention is higher than that in wild types which contain no foreign AGL21 and/or homologue proteins thereof.

As used herein, the term “foreign” means that AGL21 protein or homologue proteins thereof is not endogenous. In other words, the AGL21 gene or homologues thereof is introduced exogenously into plants.

In this invention, the AGL21 or homologue proteins thereof may be mutated as long as the mutants can promote the lateral root growth when they are expressed in plants.

The transgenic plant according to the present invention can be prepared by introducing the gene or recombinant vector into the target plant. In the present invention, “gene introduction” refers to introduction of the target gene into a cell of the host plant via, for example, a conventional gene engineering technique, so that the gene can be expressed therein. The introduced gene may be incorporated into the genomic DNA of the host plant or may be present while remaining contained in a foreign vector.

The gene or recombinant vector can be adequately introduced into a plant via a variety of reported and established techniques. Examples thereof include the Agrobacterium method, the PEG-calcium phosphate method, electroporation, the liposome method, the particle gun method, and microinjection. The Agrobacterium method may employ a protoplast, a tissue section, or a plant itself (the in planta method). When a protoplast is employed, the protoplast is cultured together with the Agrobacterium (Agrobacterium tumefaciens or Agrobacterium rhizogenes) having a Ti or Ri plasmid, or it is fused with a spheroplasted Agrobacterium (the spheroplast method). When a tissue section is employed, Agrobacterium is allowed to infect a leaf section (a leaf disc) of an aseptically cultivated target plant or a callus (an undifferentiated cultured cell). When the in planta method that utilizes seeds or plants is employed, i.e., a method that is not carried out via tissue culture with the addition of phytohormones, Agrobacterium can be directly applied to water absorptive seeds, seedlings, potted plants, and the like. Such plant transformation can be carried out in accordance with a description of a general textbook, such as “Experimental protocols of model plants (New edition), Shimamoto, K. and Okada, K (e.d.), From Genetic engineering to genomic analysis, 2001, Shujunsha.”

Whether or not the gene has been incorporated into the plant can be confirmed via PCR, Southern hybridization, Northern hybridization, Western blotting, or other means. For example, DNA is prepared from a transgenic plant, an AGL21 gene-specific primer is designed, and PCR is then carried out. After PCR has been carried out, the amplification product is subjected to agarose gel electrophoresis, polyacrylamide gel electrophoresis, or capillary electrophoresis and stained with ethidium bromide, a SYBR Green solution, or the like, thereby allowing detection of the amplification product as a band. Thus, transformation can be confirmed. Alternatively, the amplification product can be detected via PCR with the use of a primer that has been previously labeled with a fluorescent dye or the like. Further, the amplification product may be bound to a solid phase such as a microplate to thereby confirm the amplification product via fluorescent or enzyme reactions. Further, the protein may be extracted from the plant cell, two-dimensional electrophoresis may be carried out to fractionate the protein, and a band of the protein encoded by the AGL21 gene may be detected. Thus, expression of the AGL21 gene that has been introduced into the plant cell; i.e., transformation of the plant, may be confirmed.

Alternatively, a variety of reporter genes, such as β-glucuronidase (GUS), luciferase (LUC), green fluorescent protein (GFP), chloramphenicol acetyltransferase (CAT), or β-galactosidase (LacZ), are ligated to the downstream region of the target gene to prepare a vector. Agrobacterium to which the aforementioned vector has been incorporated is used to transform a plant in the same manner as described above, and the expression of the reporter gene is assayed. Thus, incorporation of the gene into the plant can be confirmed.

In the present invention, monocotyledonous plants or dicotyledonous plants may be used for transformation. Examples of such land plants include, but are not limited to, mosses, ferns, gymnosperm and angiosperm (including dicotyledonous plants, monocotyledonous plants, tree plants). Specifically, examples of plants include species belonging to orders such as Jungermanniales, Marchantiales, Eubryales, Filicales, Cycadales, Ginkgoales, Taxodiales, Pdocarpales, Ephedrales, Magnoliales, Laurales, Capparales, Fabales, Poales, Uricales, Fagales, Caryophyllales, Theales, Salicales, Ericales, Rosales, Myrtales, Sapindales, Apiales, Saponales, Lamiales and Asterales, and more specifically, include species such as Alabidopsis thaliana, Brassica napus, Brassica oleracea var. italica, Raphanus sativus L., Brassica oleraceae var. botrytis, Brassica oleracea var. capitata, Brassica rapa var. glabra, Oryza sativa, Triticum aestivum, Hordeum vulgare, Zea mays, Glycine max, Lotus corniculatus var. japonicus, Solanum lycopersicum, Solanum melongena, Solanum tuberosum L., Allium fistulosum, Allium cepa, Allium sativum, Spinacia oleracea, Saccharum officinarum, Eucalyptus, Populus, Elaeis gunineensis, Wasabia japonica, Allium tuberosum, etc.

In the present invention, examples of plant materials to be transformed include: plant organs, such as a stem, leaf, root, seed, embryo, ovule, ovary, and shoot apex; plant tissues, such as anther or pollen, and sections thereof; undifferentiated calluses; and cultured plant cells such as protoplasts prepared by removing cell walls via enzyme processing. When the in planta method is employed, water absorptive seeds or a whole plant can also be used.

A transgenic plant in the present invention refers to a whole plant, a plant organ (e.g., a leaf, petal, stem, root, grain, or seed), a plant tissue (e.g., the epidermis, phloem, parenchyma, xylem, or vascular bundle), or a cultured plant cell (e.g., callus).

After a cultured plant cell is to be transformed, the transformed callus or tissue can be selected for selectable marker (e.g., by culturing them in a medium containing antibiotic) or reporter (e.g., by detecting a fluorescence). An organ or individual may be regenerated from the obtained transformed cell via conventional tissue culture techniques. For example, the callus can redifferentiate into seedlings on a redifferentiation medium. The tissue may be transformed directly, or alternatively protoplasts may be prepared from the tissue, followed by induction of calli, which are subsequently redifferentiated into seedlings. After the roots are developed, the seedlings are transferred to soil for reproduction of plant. From the reproduced plant, seeds are collected in order to obtain transformed plants (or transgenic plants). A person skilled in the art can easily carry out such procedures via a common technique that is known as a method of regenerating a plant from a plant cell. For example, a plant can be regenerated from a plant cell in the following manner.

At the outset, when plant tissues or protoplasts are used as plant materials to be transformed, they are cultured in a callus-forming medium that has been sterilized with the addition of, for example, inorganic elements, vitamins, carbon sources, saccharides as energy sources, or plant growth regulators (plant hormones, such as auxin, cytokinin, gibberellin, abscisic acid, ethylene, or brassinosteroid), and indeterminately proliferating dedifferentiated calluses are allowed to form (hereafter, this process is referred to as “callus induction”). The thus formed calluses are transferred to a fresh medium containing plant growth regulators, such as auxin, and then further proliferation takes place (i.e., subculture).

Callus induction is carried out on a solid medium such as agar, and subculture is carried out in, for example, a liquid medium. This enables both cultures to be carried out efficiently and in large quantities. Subsequently, the calluses proliferated via the aforementioned subculture are cultured under adequate conditions to induce redifferentiation of organs (hereafter referred to as “induction of redifferentiation”), and a complete plant is finally regenerated. Induction of redifferentiation can be carried out by adequately determining the type and quantity of each ingredient in the medium, such as plant growth regulators such as auxin and carbon sources, light, temperature, and other conditions. Such induction of redifferentiation results in formation of adventitious embryos, adventitious roots, adventitious buds, adventitious shoots, and the like, which further leads to growth into complete plants. Alternatively, such items may be stored in a state that corresponds to conditions before they become complete plants (e.g., encapsulated artificial seeds, dry embryos, or freeze-dried cells and tissues).

In this invention, progeny of the transformed plants is also encompassed. Progeny includes second generation, third generation, and further subsequent generations. The progeny plant may generally be obtained via sexual reproduction or asexual reproduction of a plant into which the gene of interest has been introduced (including a plant regenerated from a transgenic cell or callus) and part of a tissue or organ of a progeny plant (e.g., a seed or protoplast). The transgenic plant of the present invention can be mass-produced by obtaining reproduction materials, such as seeds or protoplasts, from plants transformed via introduction of the AGL21 gene and cultivating or culturing the same.

In the thus-obtained transgenic plant, the nuclear DNA content in the plant cell increases via expression of the AGL21 gene. As a result, breeding of the enlarged transgenic plant of interest can be realized. The present invention, accordingly, provides a method comprising introducing the AGL21 gene or a homolog gene thereof into a plant and causing the same to overexpress in the plant, thereby enlarging the entire plant or a part thereof.

### EXAMPLES

Hereafter, the present invention is described in greater detail with reference to the following examples, although the technical scope of the present invention is not limited thereto.

### <Material and Method>

The materials and the methods employed in the examples below are as follows.

### (Plant Materials and Growth Conditions)

For the phenotypic analysis, Arabidopsis (Arabidopsis thaliana) plants were grown at 22° C. under continuous light with the light intensity of 40 μE m⁻² s⁻¹. Seeds were sterilized, imbibed in water for 3 days, and sown on agar plates set vertically for the observation of root growth phenotypes. The agar plates were prepared with basal mineral elements (Naito et al., 1994), 1% (w/v) agar and 1% (w/v) sucrose. Nitrate-less medium was prepared by replacing 3 mM KNO₃ and 2 mM Ca(NO₃)₂ in the medium with equimolar amounts of KCl and CaCl₂, respectively. KNO₃ and L-Gln were added as nitrogen source at described final concentrations.

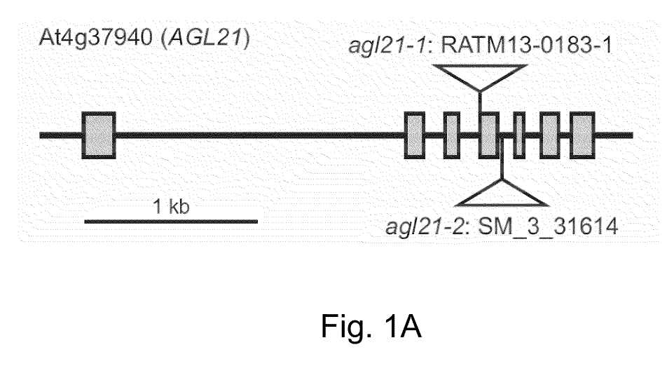

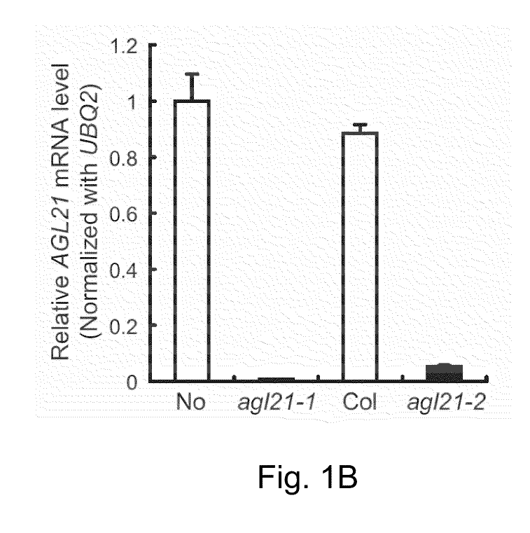

The agl21-1 mutant (RATM13-0183-1) and agl21-2 mutant (SM_{—}3_{—}31614) were obtained from RIKEN BioResource Center and John Innes Centre, respectively. The agl21-1 and agl21-2 mutants derive from the Ds insertion line collection (Kuromori et al., 2004) in ecotype Nossen background, and dSpm insertion line collection (Tissier et al., 1999) in ecotype Columbia background, respectively. The lines having homozygous insertions of transposons in AGL21 (FIG. 1) were isolated by PCR, backcrossed once to the background ecotypes, and used for the phenotypic analysis.

### (Real-Time RT-PCR)

Total RNA was extracted using the RNeasy Plant Mini Kit (Qiagen), and treated with DNase I (Invitrogen). Reverse transcription was carried out using OmniScript reverse transcriptase (Qiagen) and oligo-d(T)₁₂₋₁₈. Real-time PCR was performed by using SYBR Premix Ex Taq (Takara) and the signals were detected with 7500 Fast Real-Time PCR System (Applied Biosystems). Ubiquitin 2 (UBQ2) (GenBank accession no. J05508) was used as an internal control for normalization of transcript levels (Maruyama-Nakashita et al., 2004). Standard curves of C_{T} values for AGL21 and UBQ2 were generated using serial dilutions of cDNAs. The amounts of AGL21 in each sample were calculated from the standard curves, and normalized by those calculated for UBQ2 to obtain the relative transcript levels of AGL21. The gene specific primer sets for AGL21 and UBQ2 are listed in Table 1.

TABLE 1
Primers for construction of transgenic plants

| Primer name | Sequence | Transgenic plant | SEQ ID No. |
|---|---|---|---|
| AGL21_-2021TOPO | CACCCACAGCAAAGATAAACACACACAATTAC | AGL21 promoter-GFP | 3 |
| AGL21_-1R | CAATTTTATCCTCTAATTGAATCTCCTCTG | AGL21 promoter-GFP | 4 |
| AGL21_1TOPO | CACCATGGGAAGAGGGAAGATTGTGATC | AGL21 overexpressor | 5 |
| AGL21_2927R | TTATTCGTTTGCTCTTGGTGGAGTG | AGL21 overexpressor | 6 |

Primers for real-time RT-PCR

| Primer name | Sequence | Target gene | SEQ ID No. |
|---|---|---|---|
| AGL21_530F | ATGTGGAGCTCTACAAGAAGGC | AGL21 | 7 |
| AGL21_684R | TTCGTTTGCTCTTGGTGGAGTG | AGL21 | 8 |
| UBQ2_144F | CCAAGATCCAGGACAAAGAAGGA | UBQ2 | 9 |
| UBQ2_372R | TGGAGACGAGCATAACACTTGC | UBQ2 | 10 |

### (Transgenic Plants)

The coding region of AGL21 was amplified from the first strand cDNA of ecotype Nossen Arabidopsis plant roots by PCR using gene specific primers (Table 1) and KOD-plus DNA polymerase (Toyobo). The amplified fragment was cloned into pENTR/D-TOPO vector (Invitrogen) and fully sequenced. The acceptor GATEWAY compatible binary vector was constructed as follows. The NheI-HindIII fragment covering the 3′-end region of nopaline synthase gene promoter, basta resistance gene coding region, and polyadenylation signal of Arabidopsis RbcS-2B gene, was cut out from pBGGN which is a variant of pBGYN (Kubo et al., 2005) and inserted between the NheI and HindIII sites in pH35GS (Kubo et al., 2005) to make the basta resistant binary vector, pB35GS. The AGL21 coding sequence in the donor vector was integrated to the GATEWAY site of pB35GS using LR clonase (Invitrogen) to obtain the 35S-AGL21 construct.

The 2021 bp promoter region of AGL21 was amplified from the genomic DNA of ecotype Nossen Arabidopsis plants by PCR using gene specific primers (Table 1) and KOD-plus DNA polymerase (Toyobo). The amplified fragment was cloned into pENTR/D-TOPO vector (Invitrogen), fully sequenced, and integrated to the GATEWAY site of a binary vector pBGGN to obtain the AGL21 promoter-GFP fusion construct.

The resulting binary plasmids were transferred to Agrobacterium tumefaciens GV3101 (pMP90) (Koncz and Schell, 1986) and transformed to Arabidopsis plants according to the floral dip method (Clough and Bent, 1998). Transgenic plants were selected on agar plates containing MS salts (Murashige and Skoog, 1962), 1% (w/v) sucrose, and 10 mg l⁻¹ basta. For the overexpression of AGL21 in agl21-1 mutant, two independent lines, #20 and #10, were used for the phenotypic analysis. Transgenic and null segregants having homozygous or no integration of the 35S-AGL21 construct, respectively, were selected from these two lines for the analysis.

### (Analysis of Root Phenotypes)

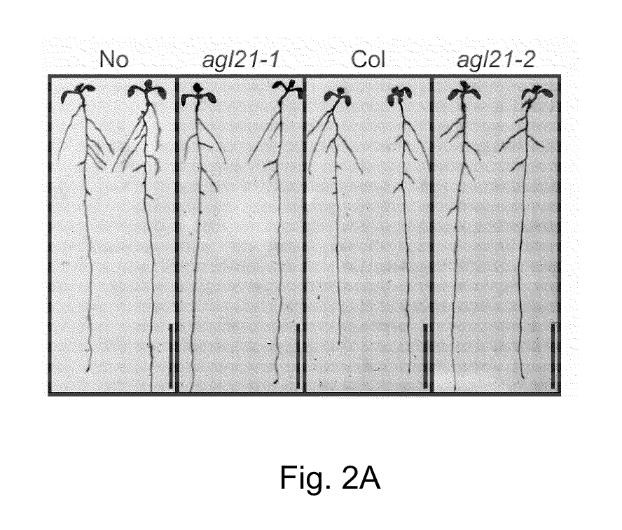

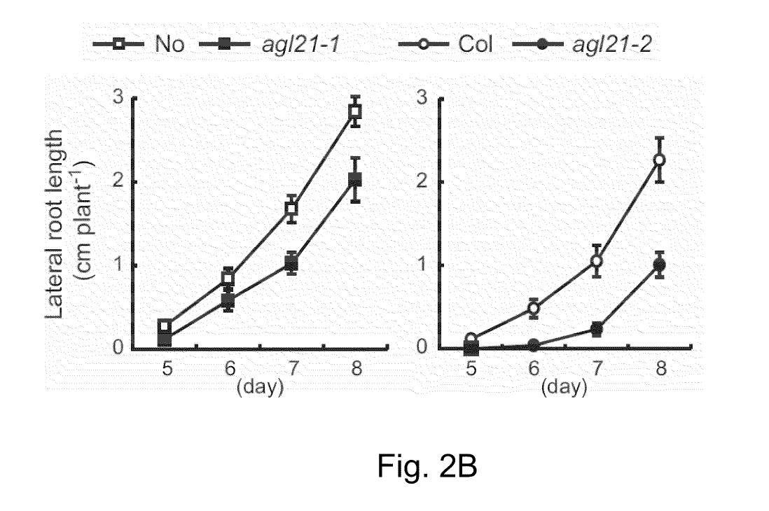

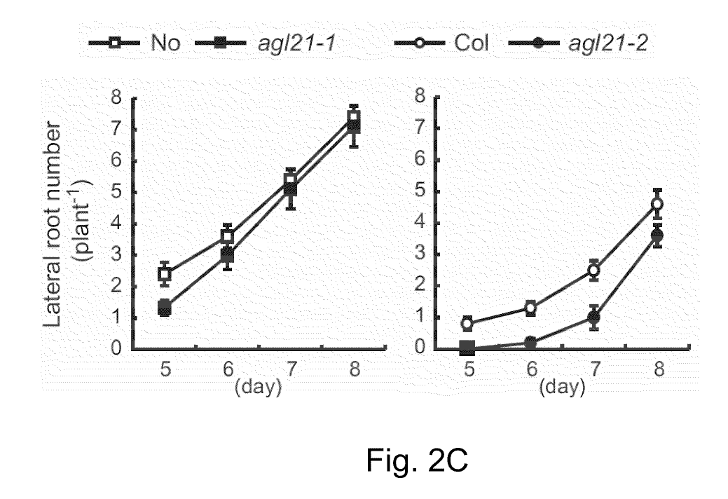

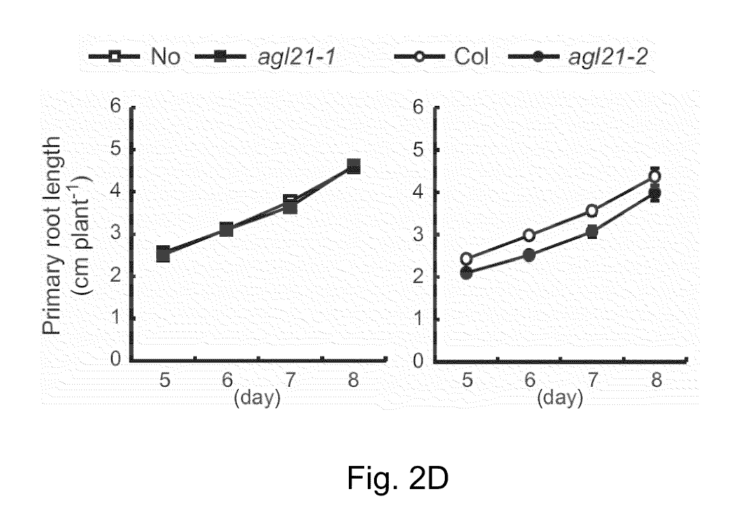

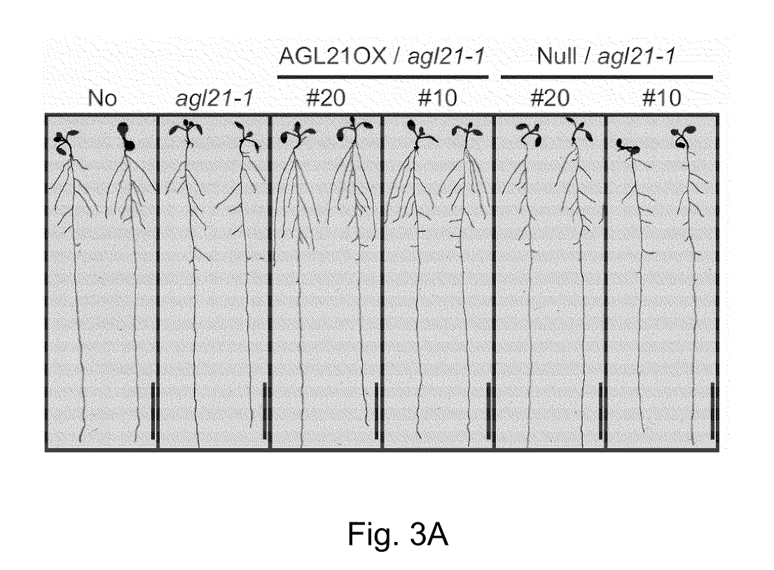

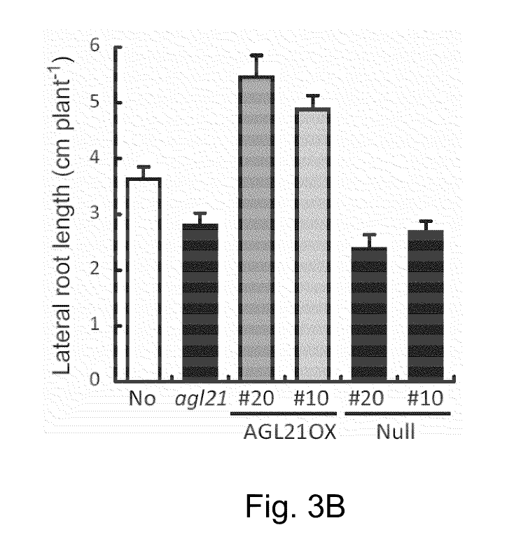

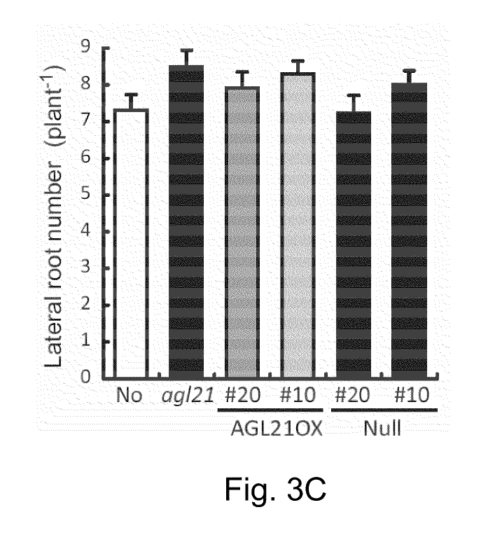

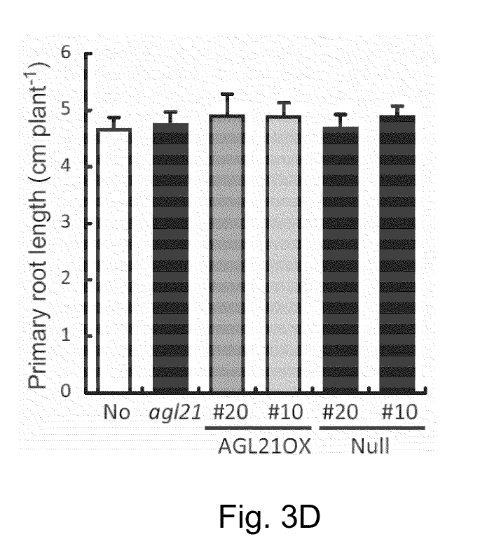

Roots were scanned using Perfection 4990 Photo transparency scanner (Epson) and root architecture was analyzed using WinRHIZO (Regent). Student's t-test (FIG. 2) and Tukey-Kramer multiple comparison test (FIGS. 3-5) were performed for the statistical analysis of phenotypic differences.

### (Microscopy)

Laser scanning confocal microscopy system FluoView500 (Olympus) was used for the analysis of localization of GFP signals. A 488-nm Ar laser and a 505-525 nm band-pass filter were used for excitation and detection of GFP. For counterstaining of cell walls, plants were stained in 10 μg ml⁻¹ propidium iodide (Sigma) for 1 min, and the fluorescence was observed under a 560 nm long-pass filter. The cross sections were constructed from Z-series confocal images using FluoView500 (Olympus).

### <Results>

### (Isolation of Knockout Mutants of AGL21)

Two independent transposon insertion lines for AGL21 (At4g37940) were identified from the collections of RIKEN (Kuromori et al., 2004) and John Innes Centre (Tissier et al., 1999). The line in Nossen background with the accession no. RATM13-0183-1 was named agl21-1, and the other line in Columbia background with the accession no. SM_{—}3_{—}31614 was named agl21-2, respectively. The identified mutants had insertions of Ds and dSpm elements in the fourth exon and fourth intron of AGL21, respectively (FIG. 1A). The mutant lines having homozygous insertions of transposons were isolated and disruption of AGL21 expression in both mutants was confirmed by real-time RT-PCR (FIG. 1B).

### (Disruption of AGL21 has a Negative Effect on Lateral Root Growth)

Root growth of the agl21 mutants was first analyzed on vertical agar plates containing 0.1 mM nitrate as the sole N source and development of the root system was monitored from the 5^{th} to 8^{th} day after sowing (FIG. 2). Under these conditions, there was a significant reduction in the rate of increase in lateral root length per plant in both agl21-1 and agl21-2 mutants compared to the wild-type plants (FIG. 2B). Initially, the numbers of visible lateral roots per plant was also reduced in the mutants (FIG. 2C), but by day 8 there was little difference in lateral root numbers between the mutants and the wild-type (FIG. 2C). Growth of the primary root in the agl21-1 mutant was the same as the wild-type and in the agl21-2 mutant it was only slightly decreased (FIG. 2D). Thus the main effect of the defect in the agl21 gene was to reduce the lateral root growth.

Using two independent AGL21 knockout lines it was shown that the inactivation of this MADS-box gene affects the growth of lateral roots under low nitrate conditions (0.1 mM) (FIG. 2C), without affecting primary root growth or the number of visible lateral roots, except in the early stage of growth (FIG. 2D). This suggests that AGL21 is a positive regulator of lateral root growth, but not primary root growth or lateral root initiation.

### (Overexpression of AGL21 Complements the Mutant Phenotype and Stimulates the Lateral Root Growth)

Transgenic plants overexpressing AGL21 were generated in the agl21-1 mutant to analyze the gain-of-function phenotypes. The coding region of AGL21 was placed under the cauliflower mosaic virus 35S promoter and transformed into the agl21-1 mutant as described in the Methods. FIG. 3A indicates the root phenotypes of two independent AGL21 overexpressing lines on agar plates containing 0.1 mM nitrate. The results indicated the overexpression of AGL21 complements the phenotype of agl21-1, as shown by the restoration of lateral root growth (FIG. 3B). The null lines segregated out from the same transformants showed short lateral root phenotypes similar to those observed in the agl21-1 mutant (FIGS. 3, A and B). Furthermore, co-segregations of the lateral root growth phenotype and the presence of 35S-AGL21 transgene in the T₂ siblings clearly indicated the observed phenotype derives from the overexpression of AGL21 (data not shown). In addition to the restoration of the mutant, the overexpression of AGL21 caused further elongation of lateral roots to exceed that in the wild-type plants (FIG. 3B). In contrast to the growth of lateral roots, the numbers of lateral roots and the growth of primary roots were not affected by overexpression of AGL21 (FIGS. 3, C and D).

(The agl21 Mutant Phenotype is Dependent on the Nitrogen Supply)

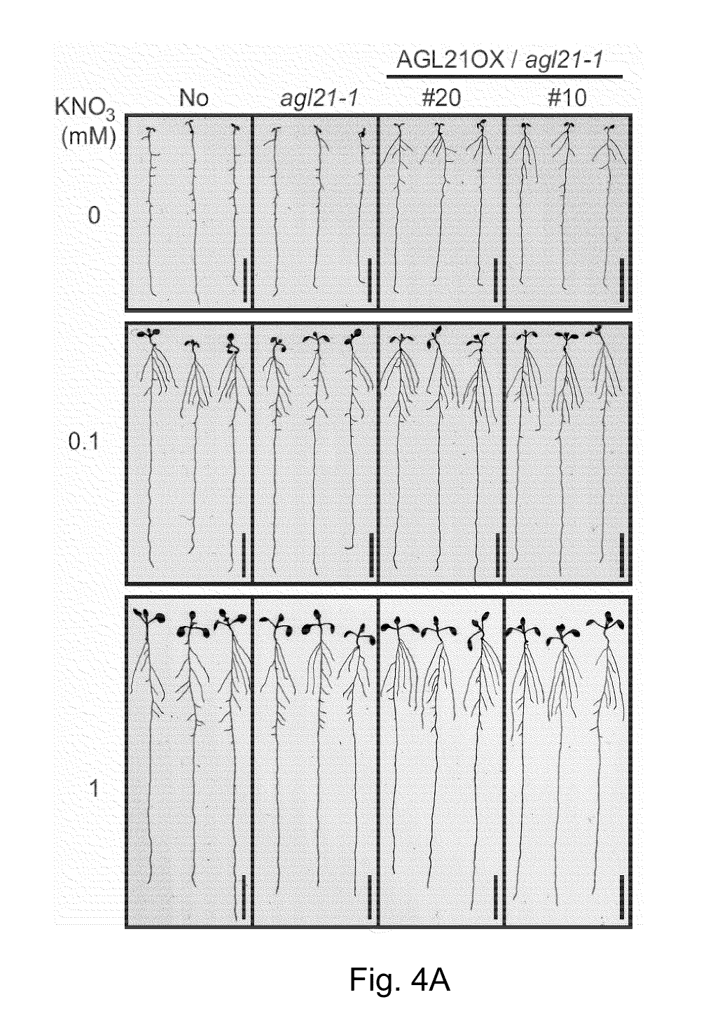

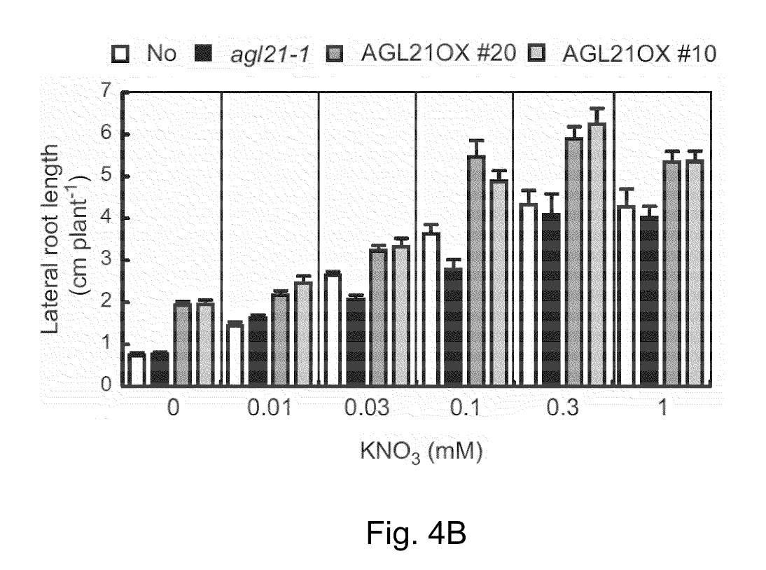

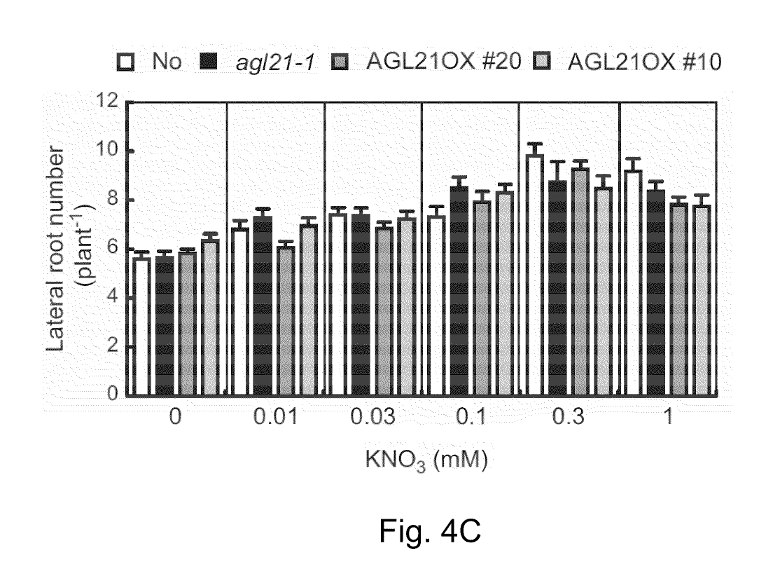

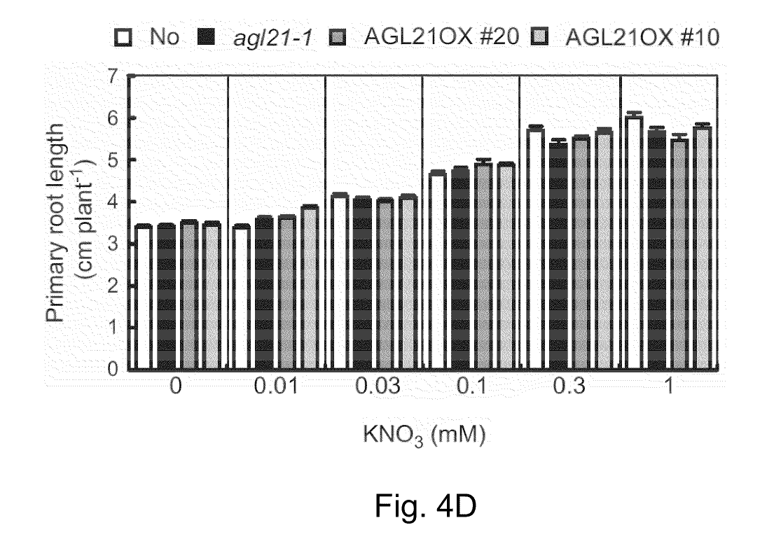

To investigate the effect of different nitrate concentrations on the agl21 mutant phenotype, wild-type (Nossen), agl21-1 mutant, and AGL21 overexpressors in agl21-1 background were cultured vertically on agar plates containing various concentrations of nitrate from 0 to 1 mM (FIG. 4A). The results indicate that the effect on lateral root growth of disrupting AGL21 expression is highly dependent on nitrate concentration (FIG. 4B). Reduced lateral root growth in agl21-1 mutant plants was seen when the external nitrate concentration was 0.03 mM or 0.1 mM. At very low nitrate concentrations (0.01 mM), or when nitrate was omitted, there was similarly no difference between agl21-1 and wild-type (FIG. 4B). In addition the defects in lateral root growth in agl21 mutant plants were less significant when higher concentrations of nitrate (0.3 mM or 1 mM) were supplied as N sources (FIG. 4B).

By contrast to the nitrate-dependent phenotype of the mutant, the two AGL21 overexpressing lines showed increased lateral root growth at all nitrate concentrations, although in percentage terms the effect was greatest in the absence of nitrate (FIGS. 4, A and B). Neither the number of visible lateral roots nor the growth of the primary roots was affected by overexpression of AGL21 (FIGS. 4, C and D).

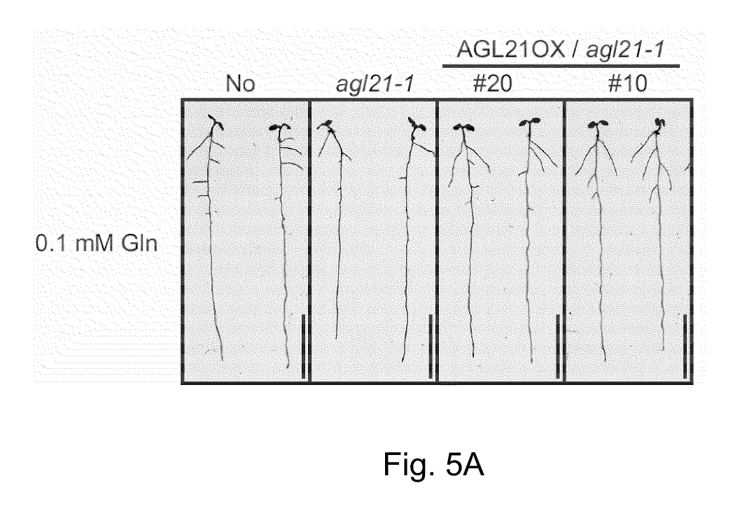

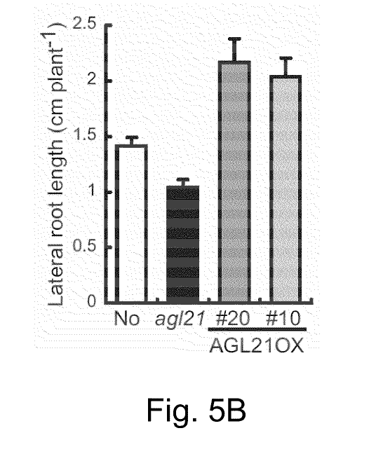

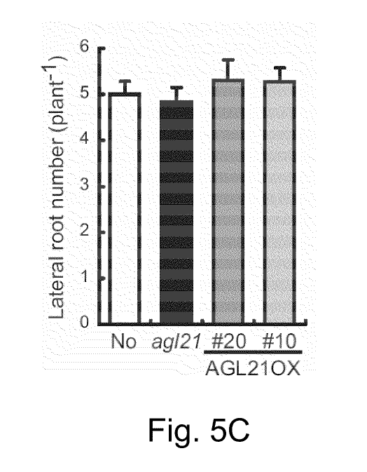

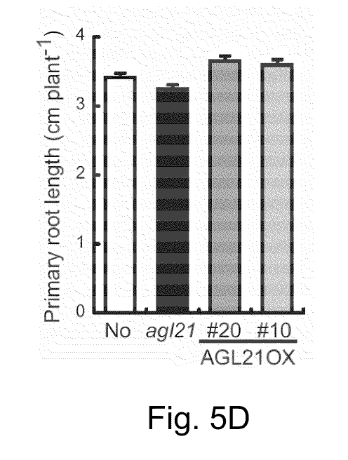

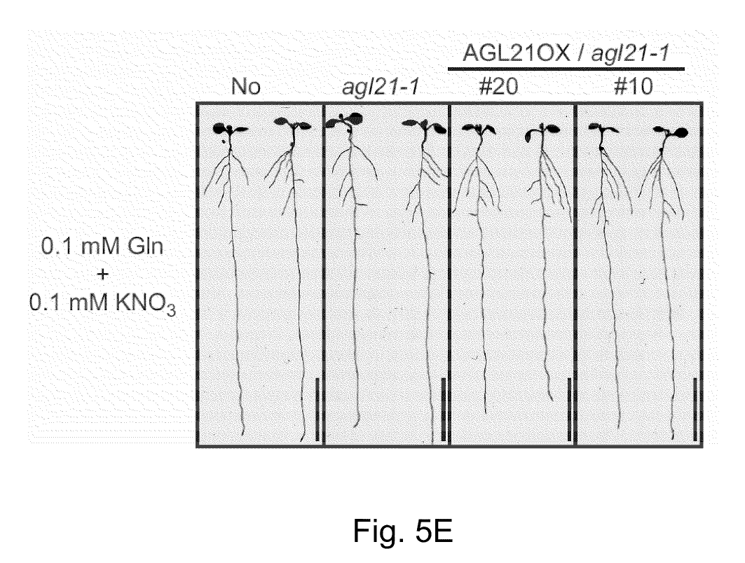

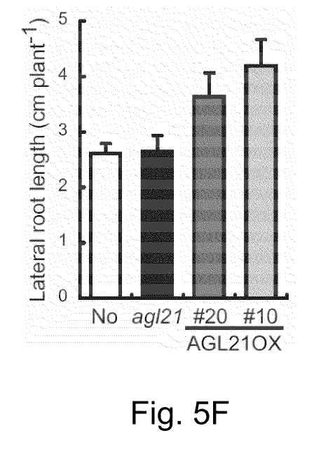

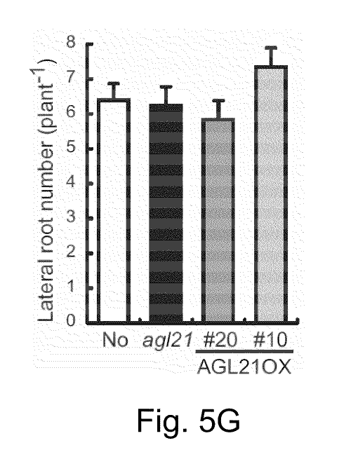

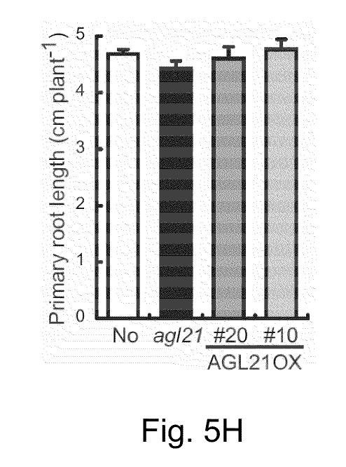

To examine whether it was the limiting availability of N or the low concentration of nitrate per se that was responsible for the appearance of the mutant phenotype, the experiment using 0.1 mM Gln as an alternative N source was prepared (FIG. 5). Compared to the zero N medium, seedling growth was substantially increased by the addition of 0.1 mM Gln (cf. uppermost panels of FIGS. 4A and 5A), presumably because of the lack of N starvation. When Gln was supplied as the sole N source (FIG. 5A), the agl21 plants had a similar root phenotype to those growing on 0.03-0.1 mM nitrate, with a significantly reduced growth of lateral roots (FIG. 5B), and no significant change in lateral root numbers (FIG. 5C) or primary root growth (FIG. 5D). However, when 0.1 mM nitrate was supplemented to the 0.1 mM Gln medium (FIG. 5E), the lateral root phenotype of agl21 mutant was lost as in the cases of higher concentrations of nitrate (>0.3 mM) (FIG. 5F). The stimulatory effect of the AGL21 overexpression on lateral root growth was evident irrespective of the absence or the presence of nitrate in the Gln medium (FIGS. 5, B and F).

### (Localization of AGL21 in Roots)

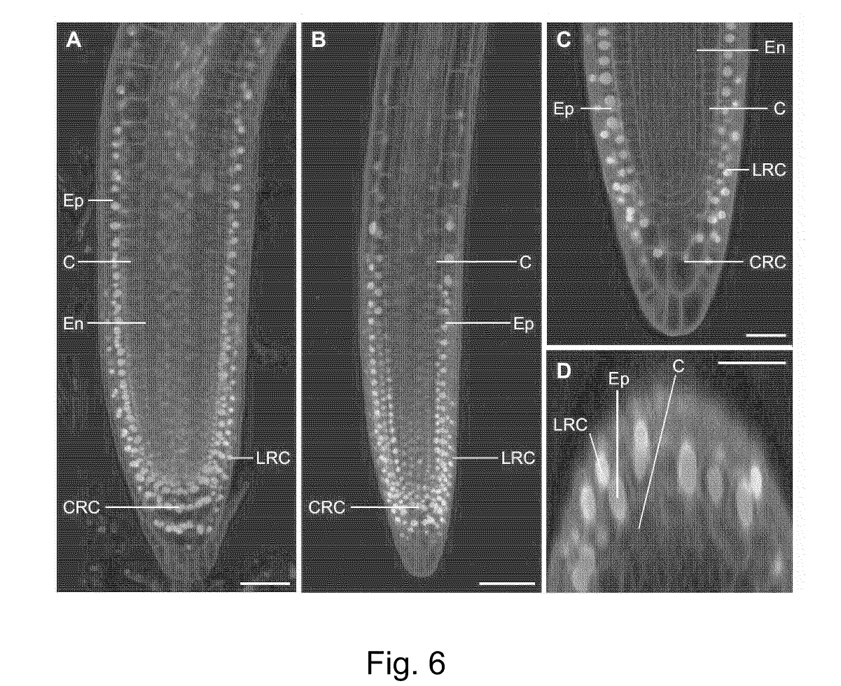

The localization of AGL21 expression in Arabidopsis seedlings was studied using transgenic plants expressing GFP under the control of AGL21 promoter. A 2021 bp 5′-region of AGL21 was fused to a nuclear-targeted GFP and transformed to ecotype Nossen Arabidopsis plants. The signals of GFP were exclusively found in roots, particularly in the tip regions of primary and lateral roots (FIGS. 6, A and B). More precisely, GFP expression was mainly restricted to the epidermal cell layers in the meristematic regions, and to the lateral root-caps and columella cells (FIGS. 6, C and D), but the signals disappeared in the elongated epidermal cells along the root axis (FIGS. 6, A and B). In contrast to the strong expression in the epidermal and root-cap cells, faint or no signals were detected in the inner cell layers (FIG. 6). The expression of AGL21-GFP construct was almost undetectable in the vascular tissue, contrary to the data from in situ hybridization (Burgeff et al., 2002).

All publications, patents, and patent applications cited herein are incorporated herein by reference in their entirety.

### REFERENCES

Burgeff C, et al. (2002) Planta 214: 365-372
Clough S J, Bent A F (1998) Plant J 16: 735-743
Crawford N M, Glass A D M (1998) Trends Plant Sci 3: 389-395
Daniel-Vedele F, et al. (1998) Curr Opin Plant Biol 1: 235-239
de Folter S, et al. (2005) Plant Cell 17: 1424-1433
Drew M C (1975) New Phytol 75: 479-490
Forde B G (2000) Biochim Biophys Acta 1465: 219-235
Forde B G, Lorenzo H (2001) Plant Soil 232: 51-68
Gan Y, et al. (2005) Planta 222: 730-742
Han P, García-Ponce B, Fonseca-Salazar G, Alvarez-Buylla E R, Yu H (2008) Plant J 55: 253-265
Hu H-C, et al. (2009) Plant J 57: 264-278
Imlau A, et al. (1999) Plant Cell 11: 309-322
Koncz C, Schell J (1986) Mol Gen Genet 204: 383-396
Kubo M, et al. (2005) Genes Dev 19: 1855-1860
Kuromori T, et al. (2004) Plant J 37: 897-905
Kutter C, et al. (2007) Plant Cell 19: 2417-2429
Little D Y, et al. (2005) Proc Natl Acad Sci USA 102: 13693-13698
Lynch J (1995) Plant Physiol 109: 7-13
Malamy J E (2005) Plant Cell Environ 28: 67-77
Malamy J E, Ryan K S (2001) Plant Physiol 127: 899-909
Marschner H (1995) Mineral Nutrition of Higher Plants, Second Ed., Academic Press, London, pp 231-255
Maruyama-Nakashita A, et al. (2004) Plant J 38: 779-789
Murashige T, Skoog F (1962) Physiol Plant 15: 473-497
Naito S, et al. (1994) Plant Physiol 104: 497-503
Parenicova L, et al. (2003) Plant Cell 15: 1538-1551
Remans T, et al. (2006a) Proc Natl Acad Sci USA 103: 19206-19211
Remans T, et al. (2006b) Plant Physiol 140: 909-921
Robinson D (1994) New Phytol 127: 635-674
Sena G, et al. (2004) Development 131: 2817-2826
Tissier A F, et al. (1999) Plant Cell 11: 1841-1852
Walch-Liu P, Forde B G (2008) Plant J 54: 820-828
Williams L E, Miller A J (2001) Annu Rev Plant Physiol Plant Mol Biol 52: 659-688
Zhang H, Forde B G (1998) Science 279: 407-409
Zhang H, et al. (1999) Proc Natl Acad Sci USA 96: 6529-6534

What is claimed is:

1. A MADS-box transcription factor which can promote the lateral root growth in a plant under nitrogen-limiting conditions.

2. The transcription factor according to claim 1, wherein the transcription factor comprises any one of the following amino acid sequences (a) to (c):
(a) an amino acid sequence defined in SEQ ID NO: 1,
(b) an amino acid sequence comprising one or more amino acid deletions, substitutions and/or additions in the amino acid sequence defined in SEQ ID NO: 1,
(c) an amino acid sequence sharing more than 50% homology with the amino acid sequence defined in SEQ ID NO: 1

3. The transcription factor according to claim 1, wherein the plant belongs to Brassicaceae, Fabaceae, Poaceae, Solanaceae, Vitaceae, Euphorbiaceae, Salicaceae or Myrtaceae.

4. A polynucleotide encoding the transcription factor of claim 1.

5. A vector comprising the polynucleotide of claim 4.

6. The vector according to claim 5, wherein the vector includes Ti plasmid, or binary plasmid.

7. A transgenic plant or a progeny thereof, comprising the polynucleotide of claim 4 or the expression vector of claim 5 or 6.

8. The transgenic plant or the progeny thereof according to claim 7, wherein the expression of the polynucleotide of claim 4 is enhanced.

9. The transgenic plant or the progeny thereof according to claim 8, wherein the expression-enhanced region is root.

10. A method for growing a plant on land with limiting nitrogen source using the transgenic plant or the progeny thereof of claim 7.

11. A method for producing a plant in which the lateral root growth is promoted, comprising the following steps:
introducing a polynucleotide of claim 4 or a vector of claim 5 or 6 into a plant, a cell or tissue thereof;
optionally, culturing the cell or tissue to reproduce plant bodies; and
selecting a plant with promoted growth of lateral roots from among the plant bodies, which are cultivated under nitrogen-limiting conditions.
